(19) United States
(12) Patent Application Publication
(10) Pub. No.: US 2026/0250724 A1
(43) Pub. Date: Aug. 27, 2026

(54) METHODS FOR CARBON-CONSERVING CELL-FREE BIOPRODUCTION

(71) Applicants: University of Washington, Seattle, WA (US); Battelle Memorial Institute, Richland, WA (US); Regents of the University of Minnesota, Minneapolis, MN (US); Georgia Tech Research Corporation, Atlanta, GA (US)

(72) Inventors: Ryan Cardiff, Seattle, WA (US); James M. Carothers, Seattle, WA (US); Diego Alba Burbano, Seattle, WA (US); Widianti Sugianto, Seattle, WA (US); Benjamin Tickman, Seattle, WA (US); Alexander Beliaev, Richland, WA (US); Vincent Noireaux, Minneapolis, MN (US); Pamela Peralta-Yahya, Atlanta, GA (US); Shaafique Chowdhury, Atlanta, GA (US)

(73) Assignees: University of Washington, Seattle, WA (US); Battelle Memorial Institute, Richland, WA (US); Regents of the University of Minnesota, Minneapolis, MN (US); Georgia Tech Research Corporation, Atlanta, GA (US)

(21) Appl. No.: 19/547,615

(22) Filed: Feb. 23, 2026

Related U.S. Application Data
(60) Provisional application No. 63/763,162, filed on Feb. 25, 2025.

(51) Int. Cl. C12P 7/46; C12N 9/00; C12N 9/02; C12N 9/04; C12N 9/06; C12N 9/10; C12N 9/78; C12N 9/88

(52) U.S. Cl. CPC C12P 7/46; C12N 9/0006; C12N 9/0008; C12N 9/0026; C12N 9/1014; C12N 9/78; C12N 9/88; C12N 9/93; C12Y 101/01037; C12Y 102/02001; C12Y 201/02001; C12Y 305/04009; C12Y 403/01017; C12Y 603/04003; C12Y 604/01001

(57) ABSTRACT

A method for cell-free bioproduction of a desired chemical compound by engineering a multi-step metabolic pathway, comprising (a) producing one or more enzymes utilized for producing a desired chemical compound from one or more DNA templates in a lysate-based cell-free expression system, and (b) producing one or more desired chemical compounds in a cell-free metabolic engineering reaction by reacting one or more substrates with the one or more enzymes in the diluted expression system.

### CROSS-REFERENCE TO RELATED APPLICATION

This application claims the benefit of U.S. Patent Application No. 63/763,162, filed Feb. 25, 2025, expressly incorporated herein by reference in its entirety.

### STATEMENT OF GOVERNMENT LICENSE RIGHTS

This invention was made with government support under Contracts DE-AC05-76RL01830, DE-AR00002387-1567, DEAC05-00OR22725, DE-AR0001514, and DE-SC0023091, awarded by the U.S. Department of Energy. The government has certain rights in the invention.

### BACKGROUND

Metabolic engineering has enabled the production of industrially relevant chemicals from renewable and low-cost one carbon (C1) feedstocks, including CO₂ and formate. The use of renewable feedstocks offers a promising alternative to petroleum-based chemical synthesis for the sustainable production of platform chemicals. However, the use of CO₂ and formate as feedstocks for common metabolic engineering chassis, such as Escherichia coli, has been limited by the low solubility of CO₂ and high formate toxicity. Novel approaches are needed to efficiently incorporate C1 feedstocks into biologically-accessible chemicals.

The use of cell-free gene expression (CFE) systems as a platform for carbon-conserving metabolic engineering has the potential to address several of the limitations of microbial based bioproduction. CFE systems are typically either prepared as a purified system, in which all the necessary components are individually purified and then reconstituted, or as a cell-based lysate. Each CFE approach has its own advantages and weaknesses, but across systems the high cost of preparation limits their potential for metabolic engineering. Lysate-based transcriptional and translational (TXTL) systems show promise for high-yield protein expression at lower costs than purified systems, while still enabling modification of reaction conditions, such as protein and chemical concentrations. CFE systems prepared from E. coli lysate contain high concentrations of cellular proteins and metabolites, including those required for transcriptional and translation, but lack the cell membrane and genomic DNA native to the cell. This allows CFE systems to be leveraged as an open reaction vessel, where plasmid or linear DNA encoding pathways of interest can be added directly to the reaction mixture.

For lysate-based metabolic engineering, there are many outstanding questions with respect to the relevant expression levels of multi-enzyme pathways, compatibility of reaction conditions for gene expression and bioproduction, and the challenges associated with the endogenous proteome of the lysate. To date, the majority of in vitro carbon-fixing pathways have been assembled using purified enzyme systems to avoid interference from native enzymes present in a lysate-based system. However, purified systems are limited by the high cost of biocatalyst preparation. While less costly to prepare than purified enzyme systems, lysate-based systems face challenges from the presence of native pathways and an inability to easily regulate endogenous enzyme activity in CFE systems. While the activity of the endogenous proteome in cell-free lysates is well documented, effective interventions are still needed to minimize diversion of flux from competing reactions in the proteome. Developing systems to understand which enzymes and metabolic branches are important focal points for diversion is crucial for cell-free metabolic engineering and bioproduction more broadly.

Several carbon-fixing or carbon-conserving bioproduction pathways have been demonstrated using purified enzyme systems. Many systems take advantage of carboxylase-dependent cycles to iteratively fix carbon into a C3 or C4 product. Carboxylase-dependent pathways are often easier to implement in vitro as they tend to avoid the need for unstable, oxygen-sensitive cofactors. However, these cyclical pathways tend to be energetically expensive in terms of NAD(P)H and ATP consumption compared to linear pathways, such as the reverse glycine cleavage pathway. However, linear C1-incorporating pathways may require the use of complex cofactors for key enzymes, such as vitamin B₁₂, ferredoxins, or rare metals. Across pathways, efficient regeneration of energy sources and cofactors is needed to improve reaction productivity and reduce costs for C1 assimilation pathways.

A need exists for novel approaches to reduce biocatalyst requirements and regulate metabolic activity to expand the potential of cell-free systems as a scalable platform for metabolic engineering. The present disclosure seeks to fulfill this need and provides further related advantages.

### SUMMARY

In one aspect, the disclosure provides methods for cell-free bioproduction of a desired chemical compound by engineering a multi-step metabolic pathway. In certain embodiments, the disclosure provides a method for cell-free bioproduction of a chemical compound by engineering a multi-step metabolic pathway, comprising:
(a) producing one or more enzymes utilized for producing a desired chemical compound from one or more DNA templates in a lysate-based cell-free expression system;
(b) producing one or more desired chemical compounds in a cell-free metabolic engineering reaction by reacting one or more substrates with the one or more enzymes in the diluted expression system,
wherein the method further comprises at least two of the following three steps:
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) producing the one or more desired chemical compounds further comprises reactions with one or more inhibitors to substantially block activity of endogenous enzymes present in the expression system that do not produce the one or more desired chemical compounds; and
(iii) wherein producing the one or more enzymes from the one or more DNA templates comprises expression of an enzymatic cofactor regeneration system to drive the production of the one or more desired chemical compounds.

In certain embodiments, the method includes steps (i) and (ii).

In other embodiments, the method includes steps (i) and (iii).

In further embodiments, the method includes steps (ii) and (iii).

In certain embodiments, the method includes steps (i), (ii), and (iii).

In a related aspect, the disclosure provides cell-free methods for the bioproduction of malate. In certain embodiments, the disclosure provides a method for cell-free bioproduction of malate, comprising:
(a) producing one or more enzymes utilized for malate production from one or more DNA templates in a lysate-based cell-free expression system; and
(b) producing malate in a cell-free metabolic engineering reaction by reacting one or more substrates with the one or more enzymes in the diluted expression system.

In certain embodiments, the method further comprises one or more of steps (i) to (iii):
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) reacting with one or more inhibitors to substantially block activity of endogenous enzymes present in the expression system that do not produce malate; and
(iii) expressing an enzymatic cofactor regeneration system to drive the production of malate.

In certain embodiments, the method includes step (i).

In other embodiments, the method includes step (ii).

In further embodiments, the method includes step (iii).

In certain embodiments, the method includes steps (i) and (ii).

In other embodiments, the method includes steps (i) and (iii).

In further embodiments, the method includes steps (ii) and (iii).

In one embodiment, the method includes steps (i), (ii), and (iii).

In certain embodiments, the method is a cell-free method for malate bioproduction via an eight-enzyme pathway.

In certain embodiments, malate is produced from formate, glycine, and bicarbonate in the following steps,
formate is coupled with tetrahydrofolate (THF) [formate assimilation via fch, ftl, and mtdA] to provide 5,10-methylenetetrahydrofolate (5,10-CH₂-THF),
5,10-CH₂-THF transfers C1 to glycine [glyA] to provide serine and recycle THF,
serine is converted to pyruvate [sds],
pyruvate is converted to oxaloacetate [pyc] with bicarbonate, and
oxaloacetate is reduced to malate [mdh] using NADH.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a schematic diagram of a representative cell-free pathway for carbon-efficient malate production in accordance with the present disclosure. In the E. coli-based cell-free system formate and glycine are converted into malate. Formate is incorporated into glycine to form serine via the four-step formate assimilation pathway. Serine is then converted to malate via the three-step reductive TCA cycle. High NADH concentrations are maintained by in situ regeneration from fdh to bias mdh flux towards malate. Gene abbreviations: ftl, formate-tetrahydrofolate ligase; fch, methenyltetrahydrofolate cyclohydrolase; mtdA, methylenetetrahydrofolate dehydrogenase (NADP); glyA, glycine hydroxymethyltransferase; sds, serine dehydratase; pyc, pyruvate carboxylase; mdh, malate dehydrogenase; fdh, formate dehydrogenase. Metabolite abbreviations: THF, tetrahydrofolate; 5,10-THF, 5,10-methylene tetrahydrofolate; GLY, glycine; SER, serine; PYR, pyruvate; OXA, oxaloacetate; MAL, malate; SUC, succinate; ICT, isocitrate; CIT, citrate; GLX, glyoxylate; FUM, fumarate; Ac-COA, acetyl-CoA.

FIGS. 2A-2C presents the analysis of the endogenous proteome of E. coli lysate-based CFE.

FIG. 2A analyzes the endogenous proteome of E. coli lysate-based CFE after dilution and spike-in pathway metabolites. CFE reactions were set up with no genes expressed. Following overnight incubation, reactions are diluted 200-fold before pathway metabolites are added or excluded (“Plain CFE”). Metabolite concentrations are analyzed over eight hours.

FIG. 2B traces the movement of malate through the TCS cycle. Flux through different TCA metabolites are traced over time for CFE reactions with or without malate added. Fumarate, succinate, and glyoxylate are shown as malate-derived metabolites with high flux.

FIG. 2C compares endogenous interconversion of pathway metabolites: malate—pyruvate; and serine—glycine. Interconversion of pathway metabolites was measured to understand the activity of endogenous enzymes in the system. For reactions with malate or pyruvate added, both malate and pyruvate are measured. For reactions with serine or glycine added, both serine and glycine are measured.

For FIGS. 2A-2C, values represent the mean±standard deviation of two technical replicates.

FIGS. 3A-3C illustrate optimization of the reductive TCA cycle in accordance with the present disclosure.

FIG. 3A illustrates that NADH regenerated with fdh pushes flux toward malate. High malate levels are maintained by co-expression of fdh and mdh. Oxaloacetate is reduced to malate via mdh using NADH as the reducing equivalent. NADH can be regenerated from NAD⁺ and formate by fdh to maintain flux towards malate and prevent the reverse reaction of mdh. Left panel: NADH concentrations in reactions containing 1 mM NAD⁺, 10 mM formate, and CFE either with or without 5 nM fdh expression. Values represent the mean±standard deviation of two technical replicates. Right panels: Malate concentrations in reactions containing 1 mM NADH, 1 mM oxaloacetate, and CFE with either 5 nM each of mdh and fdh or just mdh. Values shown represent one technical replicate.

FIG. 3B illustrates that pyc expression results in diversion of oxaloacetate flux to TCA intermediates. Top panels: Oxaloacetate and acetyl-CoA, an allosteric activator for pyc, can be fed into the TCA cycle via the irreversible citrate synthase (CS) enzyme. Entry into TCA can be inhibited by addition of the small molecule CS inhibitor, hydroxycitrate (HCT). Citrate (bottom, left) and succinate (bottom, right) concentrations are shown over time for reactions with or without pyruvate carboxylase (pyc) expressed. All reactions are diluted 200-fold for biosynthesis. Reactions contain 1 mM pyruvate, 10 mM HCO₃⁻, 1 mM ATP, 2 mM Mg(CH₃COO)₂, 1 mM acetyl-CoA, 100 μM biotin, 1 mM NADH, and 10 mM formate. −pyc reactions express 1 nM of mdh and fdh, +pyc reactions also express 5 nM of pyc. Values shown represent one technical replicate.

FIG. 3C illustrates CFE dilution and TCA inhibition enables carbon-efficient malate production from pyruvate. pyc incorporates one CO₂ equivalent into pyruvate to form oxaloacetate and requires ATP, Mg²⁺, biotin, and acetyl-CoA for activation. Malate concentrations were measured after four hours for reactions with different conditions. DNA for pathway enzymes, additional CFE dilution, and the inhibitor hydroxycitrate were changed between conditions. All reactions contain 1 mM pyruvate, 10 mM HCO₃, 10 mM HCT, 1 mM acetyl-CoA, 100 μM biotin, 1 mM ATP, 2 mM Mg(CH₃COO)₂, 1 mM NADH, and 10 mM formate. Reactions contain CFE with or without 1 nM mdh, 1 nM fdh, and 5 nM pyc. Values represent the mean±standard deviation of three technical replicates. For all panels, statistical significance was calculated using two-tailed unpaired Welch's t-tests. Asterisks indicate a statistically significant difference (*: p-value <0.05, **: p-value <0.005).

FIGS. 4A-4E characterize serine deaminase (sda) function in lysate-based CFE.

FIG. 4A illustrates screening of iron-sulfur dependent serine deaminase (sda) variants in lysate-based CFE. Iron sulfur-dependent proteins exhibited no activity in standard CFE reaction conditions. Left panel: Isoforms from E. coli, B. subtilis, M. smegmatis, and L. pneuomophila were chosen from the literature based on favorable kinetic values and previous expression in E. coli. Right panel: Reaction conditions were screened to try to rescue activity of iron-sulfur enzymes in CFE. Iron was added to assist formation of the iron-sulfur cluster, and dithiothreitol or ascorbic acid were added to create a reducing environment. All genes were expressed at 5 nM and chemicals added at 500 μM. Values shown represent one technical replicate.

FIG. 4B illustrates that pyruvate production is enabled using a promiscuous threonine deaminase (tdcB) from E. coli. tdcB belongs to the class of PLP-utilizing deaminases, avoiding the need for iron-sulfur proteins. PLP-dependent tdcB showed promiscuous activity with serine. Pyruvate concentrations are shown for reactions either with or without serine and tdcB. All reactions contain 100 μM PLP and 1 mM AMP. Values represent the mean±standard deviation of three technical replicates.

FIG. 4C illustrates malate production from serine through the carbon-conserving rTCA cycle in accordance with the present disclosure: serine is converted to malate using a four-enzyme pathway in which tdcB converts serine to pyruvate; pyruvate is carboxylated into oxaloacetate via pyc; and oxaloacetate is then reduced to malate by mdh, with cofactor regeneration from fdh to drive flux towards malate. Malate concentrations are shown for eight hour reactions containing combinations of tdcB and/or pyc, with all reactions containing fdh and mdh. Values represent the mean±standard deviation of three technical replicates.

FIG. 4D illustrates that identification of an isoform via comparative sequence analysis removes the need for additional cofactors and reduces pathway incompatibilities. AMP binding sites found for the tdcB enzyme from S. typhimurium are compared against the E. coli isoform sequence and the sds sequence.

FIG. 4E compares sds and tdcB for production of pyruvate (left) and malate (right): sds showed improved pyruvate and malate production of tdcB. Left panel: Use of the sds isoform shows improved pyruvate production relative to tdcB in the absence of AMP. Pyruvate concentrations are shown for four-hour reactions containing 1 mM serine, 100 μM PLP, and either with or without 1 mM AMP. Values represent the mean±standard deviation of two technical replicates. Right panel: Comparison of tdcB and sds for serine to malate conversion via the four-enzyme pathway. Data shown for tdcB and sds are reproduced from FIGS. 4C and 5B, respectively. Values represent the mean±standard deviation of three technical replicates.

For FIGS. 4A-4E, statistical significance was calculated using two-tailed unpaired Welch's t-tests. Asterisks indicate a statistically significant difference (*: p-value <0.05, **: p-value <0.005).

FIGS. 5A and 5B illustrate the conversion of glycine to malate in accordance with the present disclosure.

FIG. 5A illustrates the THF-dependent carbon incorporation into glycine. C1-incorporation into glycine, mediated by a THF intermediate. 5,10-methylenetetrahydrofolate (5,10-CH₂-THF) acts as a C1 donor to convert glycine and water into serine, transforming 5,10-CH₂-THF back to THF in the process. This process is catalyzed by the PLP-dependent glyA enzyme. Serine concentrations are shown for reactions containing 1 mM glycine, 1 mM 5,10-CH₂-THF, 100 μM PLP in conditions with and without 5 nM glyA expression. Values represent the mean±standard deviation of two technical replicates.

FIG. 5B illustrates a multi-enzyme pathway enables carbon-efficient malate production from glycine in CFE. The pathway consists of five enzymatic steps, including in situ NADH regeneration. Left panel: Malate concentrations are shown for multi-step reactions with different starting compounds either with or without the pathway expressed. Right panel: CO₂ conserved through the introduction of the rTCA pathway is shown for pyruvate to malate conversion. CO₂ conserved is estimated using the pathway-dependent conversion and assuming a fixed reaction stoichiometry between malate produced and CO₂ equivalents conserved. Pathway-dependent conversion represents the added conversion efficiency above the background level from the pathway enzymes. The calculations assume that for each pyruvate converted to malate, a maximum of 1 CO₂ equivalent can be fixed from bicarbonate via rTCA, and a maximum of 2 CO₂ equivalents can be released via the oxidative TCA cycle. For both panels, reactions contain all necessary chemical cofactors for the five-step pathway. Values represent the mean±standard deviation of three technical replicates.

For FIGS. 5A and 5B, statistical significance was calculated using two-tailed unpaired Welch's t-tests. Asterisks indicate a statistically significant difference (*: p-value <0.05, **: p-value <0.005).

FIGS. 6A and 6B illustrate that regeneration of THE cofactor drives carbon-efficient malate production.

FIG. 6A illustrates an enzymatic pathway for formate assimilation into THF to form 5,10-CH₂-THF in accordance with the present disclosure. The pathway consists of three enzymes and utilizes one ATP and one NADPH equivalent. THF can be recycled back into 5,10-CH₂-THF after C1 transfer to glycine. 5,10-CH₂-THF concentration is shown for reactions fed with THE, formate, NADPH, and ATP. Reactions contain CFE either with or without the formate assimilation pathway. Values represent the mean±standard deviation of three technical replicates.

FIG. 6B illustrates carbon-efficient cell-free conversion of glycine and formate to malate. The pathway consists of eight enzymes for THF recycling, NADH regeneration, and malate synthesis. Two carbon equivalents are incorporated into malate. Malate concentration is shown for reactions fed with all necessary chemical cofactors and starting from formate and glycine. Reactions contain CFE either with or without the full pathway. In this experiment, cell-free bioproduction reactions were run at 250 μL volume. Values represent the mean±standard deviation of six technical replicates.

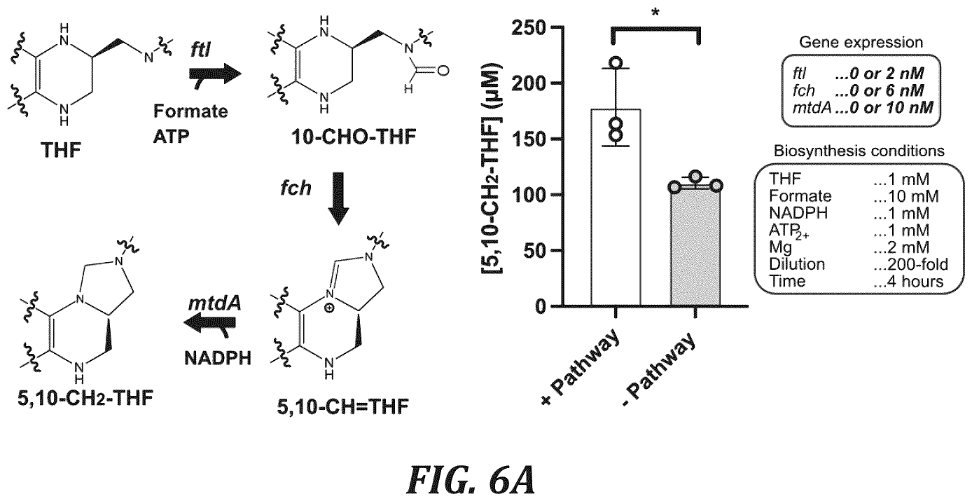

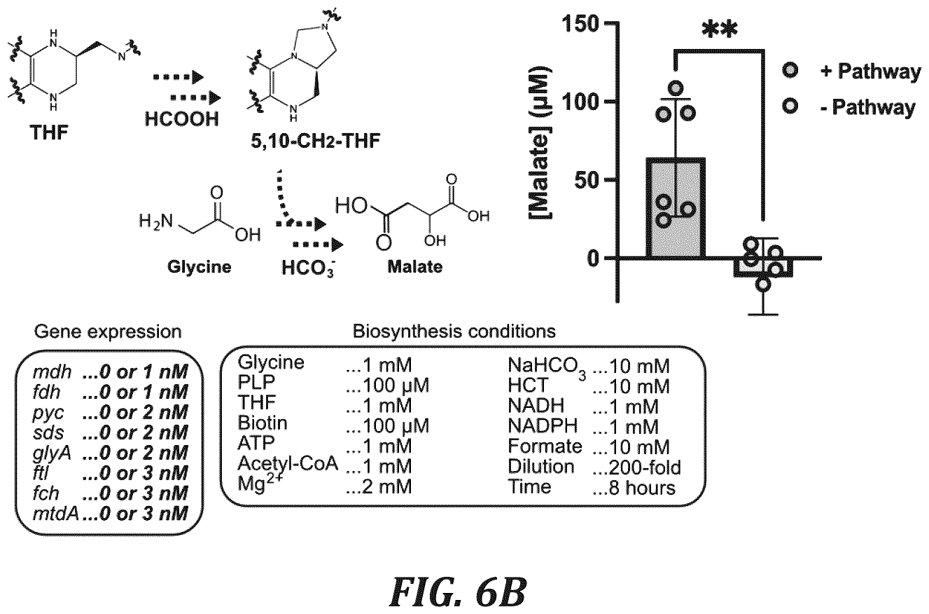

For FIGS. 6A and 6B, statistical significance was calculated using two-tailed unpaired Welch's t-tests. Asterisks indicate a statistically significant difference (*: p-value <0.05, **: p-value <0.005).

### DETAILED DESCRIPTION

The present disclosure provides a platform for the carbon-conserving bioproduction of malate in a lysate-based CFE system. Microbial bioproduction of the industrial di-acid malate requires re-routing flux through either the oxidative or reductive TCA (rTCA) cycle. The rTCA cycle represents the most carbon-efficient route, with a theoretical yield of 1 mol malate/mol pyruvate with an extra carbon coming from bicarbonate; compared to a theoretical yield of 0.5 mol malate/mol pyruvate through the oxidative TCA cycle, which features two decarboxylation steps. Microbial production through either pathway requires extensive genome engineering to introduce knockouts that will redirect metabolic flux towards malate accumulation. This often leads to cells that have significant growth defects, as the knocked-out genes are typically involved in the TCA cycle or central carbon metabolism. Cell-free bioproduction has the potential to overcome these limitations by decoupling growth and bioproduction. The present disclosure provides a cell-free platform for malate production through the carbon-efficient rTCA pathway.

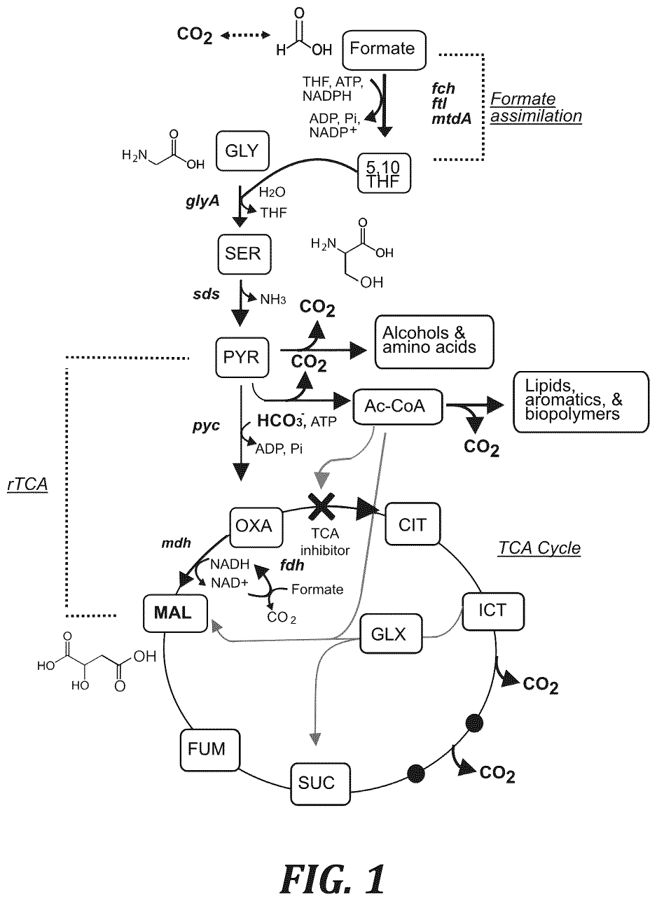

The presented pathway incorporates two carbon-fixation steps to generate the C4 di-acid malate from the C2 precursor glycine and C1 inputs formate and bicarbonate (FIG. 1). This system utilizes steps from both the formate assimilation pathway and reductive TCA cycle (rTCA). In the formate assimilation pathway, formate is first coupled with tetrahydrofolate (THF) to ultimately generate the one-carbon donor, 5,10-methylenetetrahydrofolate (5,10-CH₂-THF). Next, 5,10-CH₂-THF transfers the C1 to glycine to synthesize serine and recycle THF. Serine is then converted to pyruvate, where another CO₂ equivalent is incorporated through pyruvate carboxylase (pyc) to generate oxaloacetate. Oxaloacetate is then reduced to malate via malate dehydrogenase (mdh) using NADH. It was found that the expression of formate dehydrogenase (fdh) to regenerate NADH helps drive metabolic flux towards malate by maintaining a high concentration of reducing equivalents.

The present disclosure provides strategies to overcome limitations of lysate-based CFE metabolic engineering to successfully implement complex pathways. In the system described herein, the one-pot metabolic engineering platform into two parts by separating the cell-free protein synthesis reaction from the bioproduction reaction, which allows for independent tuning conditions for each process. By diluting protein synthesis reactions, the volumetric efficiency of the bioproduction reaction is improved and reduces the relative activity from endogenous enzymes in the lysate. In the system, metabolic flux from competing pathways was blocked by the addition of small-molecule inhibitors directly to the bioproduction reaction. In addition to improving efficiency, this approach minimizes carbon loss through the TCA cycle without requiring engineering of the underlying strain. Together, these developments enable malate production from formate and glycine with a yield on glycine of 6.4% in an eight-hour biosynthesis reaction. The present disclosure establishes the capabilities of CFE systems as a platform for combined protein synthesis and metabolic engineering, enabling carbon-conserving bioproduction of platform chemicals.

In one aspect, the disclosure provides methods for cell-free bioproduction of a desired chemical compound by engineering a multi-step metabolic pathway. In certain embodiments, the disclosure provides a method for cell-free bioproduction of a chemical compound by engineering a multi-step metabolic pathway, comprising:
(a) producing one or more enzymes utilized for producing a desired chemical compound from one or more DNA templates in a lysate-based cell-free expression system;
(b) producing one or more desired chemical compounds in a cell-free metabolic engineering reaction by reacting one or more substrates with the one or more enzymes in the diluted expression system,
wherein the method further comprises at least two of the following three steps:
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) producing the one or more desired chemical compounds further comprises reactions with one or more inhibitors to substantially block activity of endogenous enzymes present in the expression system that do not produce the one or more desired chemical compounds; and
(iii) wherein producing the one or more enzymes from the one or more DNA templates comprises expression of an enzymatic cofactor regeneration system to drive the production of the one or more desired chemical compounds.

In certain embodiments, the method includes steps (i) and (ii).

In other embodiments, the method includes steps (i) and (iii).

In further embodiments, the method includes steps (ii) and (iii).

In certain embodiments, the method includes steps (i), (ii), and (iii).

The cell-free bioproduction method includes the use of an expression system for synthesizing enzymes that are subsequently used for producing the desired chemical compounds in a multi-step (multi-enzyme) pathway. The cell-free bioproduction method described herein can be carried out in any suitable vessel that can contain the expression system and the substrates and components for the bioproduction step. In the expression system, one or more enzymes, which are utilized for producing a desired chemical compound, are produced from one or more DNA templates in a lysate-based cell-free expression system. The one or more DNA templates is the DNA used to express the enzymes to be used in bioproduction step and that these expressed enzymes are those that define the multi-step (multi-enzyme) metabolic pathway.

In certain embodiments, the method for cell-free bioproduction of a chemical compound by engineering a multi-step (multi-enzyme) metabolic pathway described herein is a linear metabolic pathway with the goal of limiting non-productive enzymatic activity from endogenous enzymes through pathway the branch points.

In certain embodiments, the one or more DNA templates comprise plasmid or linear DNA.

In certain embodiments, the lysate-based cell-free expression system is a cell lysate comprising cellular proteins and metabolites and is substantially free of cell membrane and genomic nucleic acid.

In certain embodiments, the lysate-based cell-free expression system is an E. coli lysate.

In certain embodiments, diluting the expression system comprises diluting from about 10-fold to about 1000-fold (e.g., 200-fold). Representative diluents include aqueous diluents including water and aqueous buffer solutions, such as HEPES, Tris, PBS, or imidazole buffered solutions.

In certain embodiments of the methods, the enzymatic cofactor regeneration system comprises expression of formate dehydrogenase (fdh) to regenerate NADH to drive metabolic flux towards the desired chemical compound by maintaining a high concentration of reducing equivalents.

In certain embodiments of the methods, the one or more DNA templates encode formate-THF ligase (ftl), methenyl-THF cyclohydrolase (fch), methylene-THF dehydrogenase (mtdA), Mdt, GcvHPL, GcvHTP, glycine hydroxymethyltransferase (glyA), pyruvate carboxylase (pyc), malate dehydrogenase (mdh), serine dehydratase (sds), polyphosphate kinase (PPK), lipoamide dehydrogenase (Lpd), lipoate-protein ligase A (LplA), serine hydroxymethyltransferase (Shmt), serine dehydratase (Sda), PCX, formate dehydrogenase (fdh), PPT, GcvH, GcvT, GcvP, and combinations thereof.

In certain embodiments of the methods, the one or more enzymes are formate-THF ligase (ftl), methenyl-THF cyclohydrolase (fch), methylene-THF dehydrogenase (mtdA), Mdt, GcvHPL, GcvHTP, glycine hydroxymethyltransferase (glyA), pyruvate carboxylase (pyc), malate dehydrogenase (mdh), serine dehydratase (sds), polyphosphate kinase (PPK), lipoamide dehydrogenase (Lpd), lipoate-protein ligase A (LplA), serine hydroxymethyltransferase (Shmt), serine dehydratase (Sda), PCX, formate dehydrogenase (fdh), PPT, GcvH, GcvT, GcvP, and combinations thereof.

In a related aspect, the disclosure provides cell-free methods for the bioproduction of malate. In certain embodiments, the disclosure provides a method for cell-free bioproduction of malate, comprising:
(a) producing one or more enzymes utilized for malate production from one or more DNA templates in a lysate-based cell-free expression system; and
(b) producing malate in a cell-free metabolic engineering reaction by reacting one or more substrates with the one or more enzymes in the diluted expression system.

In certain embodiments, the method further comprises one or more of steps (i) to (iii):
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) reacting with one or more inhibitors to substantially block activity of endogenous enzymes present in the expression system that do not produce malate; and
(iii) expressing an enzymatic cofactor regeneration system to drive the production of malate.

In certain embodiments, the method includes step (i).

In other embodiments, the method includes step (ii).

In further embodiments, the method includes step (iii).

In certain embodiments, the method includes steps (i) and (ii).

In other embodiments, the method includes steps (i) and (iii).

In further embodiments, the method includes steps (ii) and (iii).

In one embodiment, the method includes steps (i), (ii), and (iii).

In certain embodiments, the method is a cell-free method for malate bioproduction via an eight-enzyme pathway. See FIG. 1.

In certain embodiments of these methods, the one or more DNA templates encode fch, ftl, mtdA, glyA, sds, pyc, mdh, and fdh. In certain embodiments of these methods, the one or more enzymes are fch, ftl, mtdA, glyA, sds, pyc, mdh, and fdh.

In methods that utilize formate, glycine, and bicarbonate as substrates, the enzymes in the pathway include fch, ftl, mtdA, glyA, sds, pyc, mdh, and fdh. In methods that start with serine as the substrate, the enzymes in the pathway include sds, pyc, mdh, and fdh. Referring to FIG. 1, fdh is preferred to be included these methods, but is not required.

As noted above, in certain embodiments of these methods, the one or more substrates are formate, glycine, and bicarbonate (formate is a substrate for fch, glycine and 5,10-CH₂-THF (an intermediate) are substrates for glyA, and bicarbonate is a substrate for pyc). However, referring to FIG. 1, it will be appreciated that the method for malate bioproduction can start at glycine (GLY), serine (SER), pyruvate (PYR), or oxaloacetate (OXA).

In certain embodiments, the one or more inhibitors include hydroxycitrate. Citrate synthase and ATP citrate lyase are inhibited by hydroxycitrate.

In certain embodiments, the enzymatic cofactor regeneration system provides in situ regeneration of NADH. NADH/NADPH regeneration is through fdh. ATP regeneration is through ppk. THF regeneration is through formate assimilation

In certain embodiments, malate is produced from formate, glycine, and bicarbonate in the following steps,
formate is coupled with tetrahydrofolate (THF) [formate assimilation via fch, ftl, and mtdA] to provide 5,10-methylenetetrahydrofolate (5,10-CH₂-THF),
5,10-CH₂-THF transfers C1 to glycine [glyA] to provide serine and recycle THF,
serine is converted to pyruvate [sds],
pyruvate is converted to oxaloacetate [pyc] with bicarbonate, and
oxaloacetate is reduced to malate [mdh] using NADH.

In certain of these embodiments, the method further comprises regenerating NADH in the oxaloacetate reduction step with formate dehydrogenase (fdh) to drive metabolic flux towards malate by maintaining a high concentration of reducing equivalents.

In certain of these embodiments, or in addition to these embodiments, the method further comprises adding an inhibitor (e.g., hydroxycitrate) to block the conversion of oxaloacetate to citrate to prevent siphoning off of oxaloacetate from malate production.

### Metabolomics Analysis of the Endogenous Metabolic Activity of the CFE

While the lysis and filtration steps in the preparation of CFE lysates remove bulky membrane proteins and genomic DNA, many native protein complexes are still present at high concentrations. The basal metabolic activity in the cell-free system was characterized by measuring the activity from endogenous enzymes in the system via targeted metabolomics.

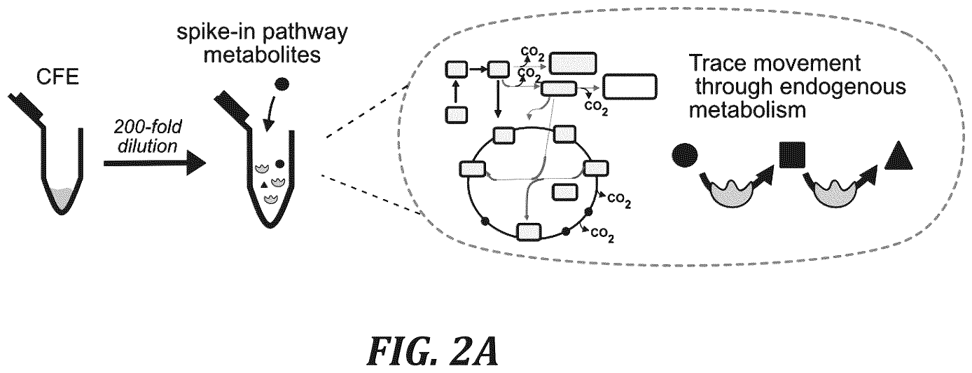

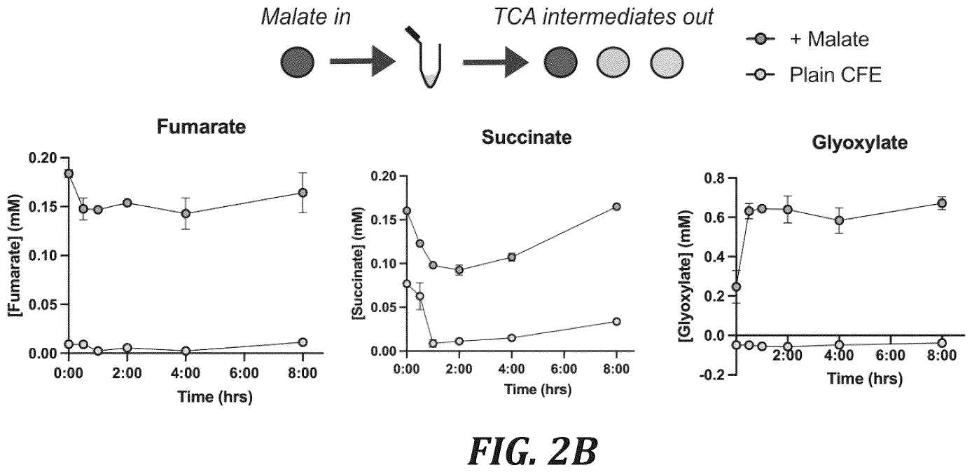

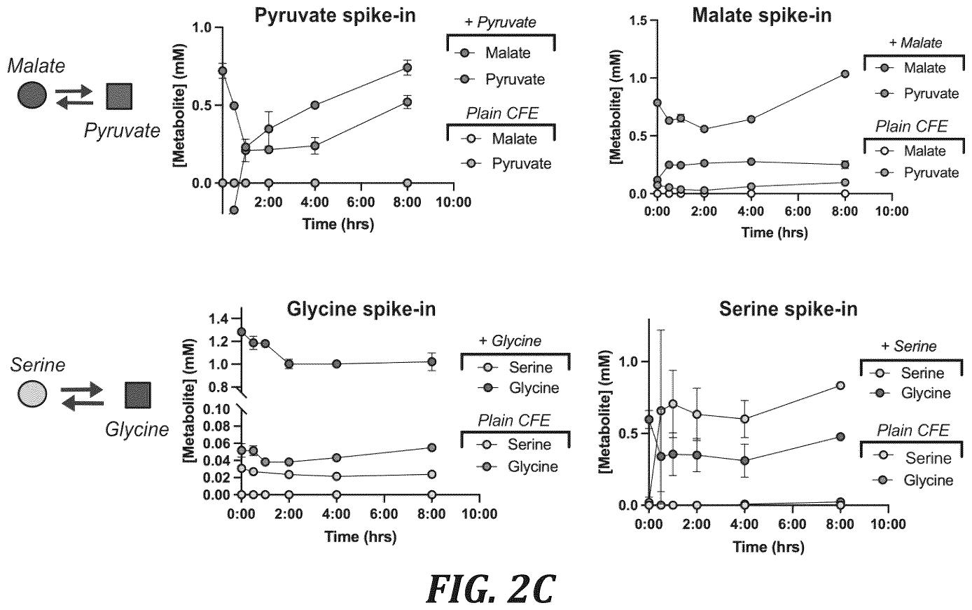

To understand the relative carbon flux as a result of the endogenous enzymes present in the cell-free lysate, 1 mM of pathway intermediates was spiked into otherwise empty, plain cell-free reactions and measured the presence of relevant compounds, including malate, pyruvate, fumarate, glyoxylate, and succinate, over time (FIG. 2A). In these experiments, the “Plain CFE” reaction represents a cell-free reaction with no pathway intermediate spiked in. It was found that, relative to a plain cell-free reaction with only water added, CFE with 1 mM malate spiked in accumulated high concentrations of fumarate, glyoxylate, succinate, and pyruvate (FIGS. 2B and 2C), indicating that TCA enzymes are highly active in the cell-free lysate. Malate was rapidly converted to other TCA intermediates. In eight hours, a molar ratio of roughly 1:1:4:4 (fumarate:succinate:glyoxylate:pyruvate) was achieved. Additionally, there is a high interconversion between pyruvate and malate, as shown by the reactions spiked with either compound having significant flux towards the other. With the addition of 1 mM glycine, serine concentrations stay roughly constant at 25 μM (FIG. 2C). In contrast, spiking 1 mM serine leads to a rapid accumulation of glycine, reaching 485 μM, or 48.5% conversion, after 8 hours. A thermodynamic analysis using Equilibrator indicated that glycine is more energetically favorable than serine (ΔG=+6.7 kJ/mol), which may explain the larger flux towards glycine. Qualitatively, the observation that spiking in serine leads to large amounts of glycine, while spiking in glycine leads to modest amounts of serine, (FIG. 2C) agrees with the thermodynamic analysis. The decreasing concentrations seen for malate and glycine early in the reaction (FIG. 2C) may be due to an initial surge in production that happens rapidly and is captured at the zero-time point, which cannot be a true zero time point due to limitations in the time required for sample preparation (roughly 5-10 minutes). Therefore, the concentration of these metabolites likely increases rapidly before the first reading, then decreases slowly. Additionally, the activity of enzymes carrying out these conversions may be affected in an in vitro cell-free system, which could lead to behaviors that do not agree with thermodynamic predictions. These results highlight the large effects of the endogenous proteome on metabolite levels in lysate-based cell-free systems.

Malate Synthesis from the Reductive TCA Cycle

To engineer a carbon-efficient pathway for malate bioproduction, malate biosynthesis was prototyped from the TCA cycle precursors oxaloacetate and pyruvate. Time-resolved measurements of malate production with cell-free expression of E. coli malate dehydrogenase (mdh) expressed from plasmid DNA with the p70 promoter was collected. It was found that in the case where mdh was overexpressed (+mdh), malate accumulated rapidly, achieving over 100% conversion within 10 minutes. Malate concentrations then rapidly decrease and reach baseline levels by 24 hours after the reaction start time in the +mdh condition. In the reaction without mdh overexpression (−mdh), a gradual increase in malate levels over time was observed. The difference between the +mdh and −mdh samples may be a result of the changes in reaction thermodynamics driven by differences in metabolite concentrations. The +mdh reaction will have higher amounts of NAD⁺ and malate early in the reaction due to the higher concentration of the mdh enzyme. This, in turn, could drive the mdh reaction in the oxidative direction and into the TCA cycle. It is worth noting that physiologically, the mdh reaction tends to run in the oxidative direction, despite the favorable thermodynamics for the reductive direction. This pull towards oxaloacetate would result in a decrease in malate titers over time. High initial concentrations of malate could also have driven flux through the reversible fumarase enzyme, leading to a decrease in malate. Thermodynamic analysis predicts that the fumarase reaction prefers the formation of fumarate when malate concentrations are roughly 10-fold higher than fumarate (ΔG=−2.3 KJ/mol).

Given that the CFE still maintains endogenous copies of TCA enzymes, the −mdh reaction may still be producing malate through the reductive mdh reaction, but at a slower rate than the +mdh reaction (as the reaction rate should be proportional to the enzyme concentration). Additionally, conversion of oxaloacetate to citrate is strongly favored thermodynamically (ΔG=−38.8 KJ/mol), which could also lead to malate production via the oxidative TCA cycle in the case where mdh is not overexpressed.

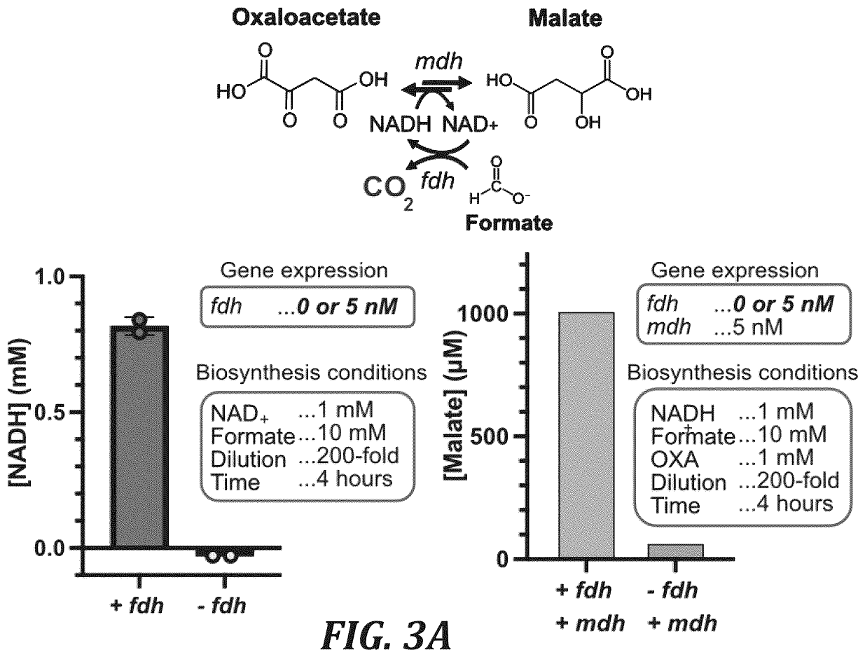

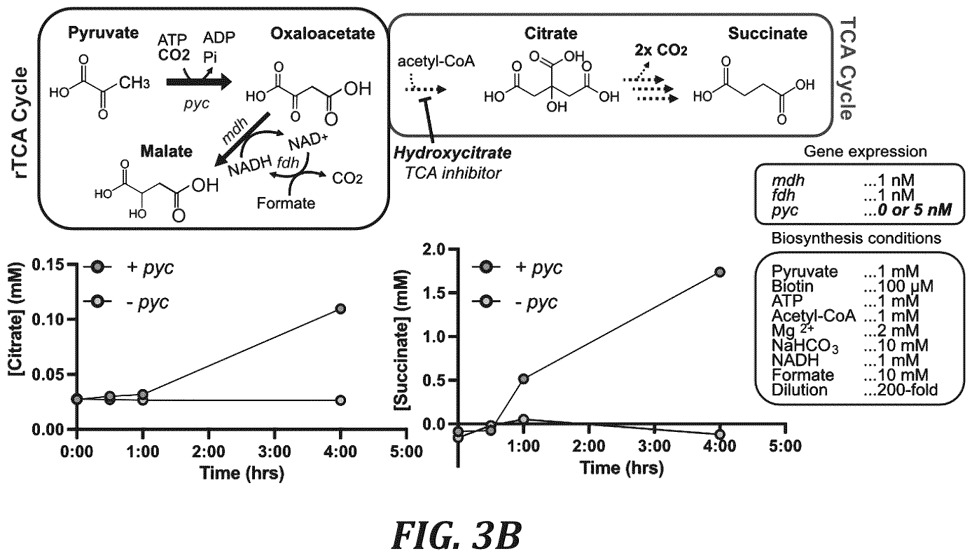

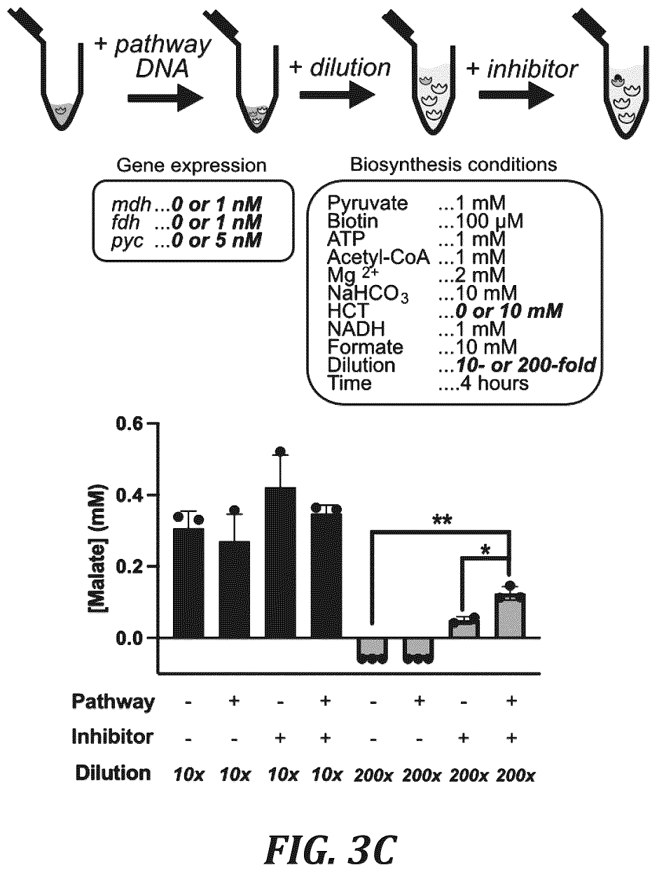

Malate synthesis was coupled with in situ NADH regeneration to allow for continuously drive flux towards malate in the CFE. To favor mdh towards malate, a NADH regeneration system was incorporated (based on a formate dehydrogenase (fdh) mutant from Starkeya novella (Biochemical and Structural Insight into the Chemical Resistance and Cofactor Specificity of the Formate Dehydrogenase from Starkeya novella. FEBSJ. 2023, 290 (17), 4238-4255), fdh can regenerate NADH and CO₂ from formate and NAD⁺). Addition of NAD⁺ and formate to CFE with fdh expressed resulted in near complete conversion of NAD⁺ to NADH (FIG. 3A). It was found that upon addition of NADH and excess formate to reactions that had co-expressed mdh and fdh, malate concentrations were stable at 1000 μM, representing full conversion efficiency, up to 24 hours (FIG. 3A).

When mdh plasmid was omitted from the CFE, malate concentrations increased slowly over the reaction lifetime rather than accumulating quickly. This is likely due to the presence of endogenous enzymes in the lysate that can natively produce malate. A proteomic analysis of E. coli cell-free lysate found that mdh and other TCA enzymes are among the most abundant enzymes in cell-free. Therefore, additional dilution of the cell-free reaction following protein expression was investigated to improve the pathway-dependent production of malate by minimizing the effects of competing endogenous enzymes.

Three different dilutions of the CFE: 10 (our standard dilution factor), 50, and 200-fold were tested. It was found that the 200-fold dilution condition accumulated more malate than the other conditions when mdh was expressed. An explanation for the differences in malate production across the different dilution factors is that dilution of the cell-free lysate is more disruptive to the activity of competing TCA enzymes than it is to the overexpressed mdh. Dilution of the cell-free lysate results in the concentration of substrates and cofactors falling below relevant levels for competing enzymes with similar kinetic values. For example, citrate synthase (CS) has a similar reported Km for oxaloacetate as malate dehydrogenase (30 and 40 μM, respectively). However, CS requires acetyl-CoA as a substrate which would be present in concentrations far below the Km in the more dilute samples. NADH and ATP also serve as allosteric inhibitors of CS. In the diluted reactions, the concentrations of NADH and ATP become much higher than the concentration of the CS enzyme, which would lead to a higher percentage of CS enzymes being bound and inhibited. Additionally, the dilution of the CFE may disrupt metabolon formation between TCA enzymes that accelerate malate loss. It was also found that the 200-fold dilution accumulated less malate than the other dilutions when mdh was not expressed, suggesting the activity of native mdh had also fallen below relevant levels in those conditions.

Oxaloacetate, the direct C4 precursor of malate in the pathway, can be generated from C3 pyruvate and CO₂ using the enzyme pyruvate carboxylase (pyc) from Rhizobium etli. The pyc enzyme is a large enzyme complex made up of three distinct domains: the ATP-consuming biotin carboxylase, carboxyltransferase, and biotin carboxyl carrier protein (BCCP) domain. The pyruvate carboxylase reaction was coupled with mdh and fdh to produce malate from the C3 precursor, pyruvate (FIG. 3B). However, when pyc activity from a 10-fold diluted CFE reaction was tested, no differential malate production in the conditions was observed with and without pyc, with both conditions producing roughly 300 μM after four hours. These results suggested that the engineered pathway may be outcompeted by the native carbon-catabolizing oxidative branch of the TCA cycle.

Oxaloacetate and pyruvate were incorporated into the oxidative TCA cycle before being converted to malate by the heterologous pathway. Oxaloacetate and acetyl-CoA could be diverted into the endogenous oxidative TCA cycle by the enzyme citrate synthase, which is native to the lysate. The pyc-containing reactions are supplemented with acetyl-CoA, an allosteric activator shown to be essential for pyc activity. It was hypothesized that greater dilutions of the CFE may improve flux through our engineered pathway, as pyruvate carboxylase has a lower reported dissociation constant for acetyl-CoA than that of citrate synthase. In the 10-fold diluted CFE condition, pyc-dependent malate production above the baseline levels generated by the endogenous lysate enzymes was not observed. In the 200-fold dilution condition, increased pyc-dependent malate production compared to the baseline was observed in the first hour of the reaction, but after four hours the accumulated malate completely disappeared from both reactions (FIG. 3C). The large spike in malate may be due to an excess of NADH initially present in the system that drives flux in the reductive direction of mdh. From kinetic data, it was found that titers of the TCA cycle intermediates citrate and succinate increase over time in the reactions containing pyc, suggesting that the malate initially produced is converted to these compounds (FIG. 3B). Malate may be converted back to oxaloacetate, where oxaloacetate is then diverted into the oxidative TCA cycle by the citrate synthase enzyme.

### Blocking TCA Flux to Improve Pathway Carbon-Conservation

To minimize the loss of carbon through the oxidative TCA cycle, both targeted chemical inhibition of citrate synthase and re-routing of flux through overexpression of the glyoxylate shunt enzymes were investigated. Hydroxycitrate (HCT) is a potent competitive inhibitor of citrate synthase and ATP citrate lyase due to its structural similarity to citrate. The ability of HCT to improve flux through the engineered pathway was tested by adding varying amounts of hydroxycitrate to cell-free reactions and measuring malate accumulation over time. In the highest HCT concentration tested (10 mM), pyc-dependent accumulation of malate was observed, reaching 212±60 μM within four hours. Additionally, the change in CO₂ concentrations was measured in the headspace of the reactions but was not found to be a reliable readout of reaction progress.

In the reactions containing HCT, it was found that concentrations of citrate and succinate remain constant relative to reactions with no pathway expressed, suggesting that flux through the oxidative TCA cycle is being reduced effectively. Additionally, accumulation of acetyl-CoA was observed in the condition with the highest HCT concentration, implying that citrate synthase inhibition can prevent oxaloacetate consumption via the TCA cycle in cell-free reactions. When combined, 200-fold dilution of the lysate and HCT inhibition lowered the background activity over 6-fold and improved pathway-dependent malate production 2.5-fold compared to 10-fold diluted reactions without TCA cycle inhibitor (p<. 05) (FIG. 3C) (Table 1). In the conditions with 200-fold dilution and HCT inhibition, high concentrations of citrate were observed that do not change depending on the presence of the pathway, indicating that the TCA cycle is blocked at this point. Additionally, no changes in succinate were observed across any of the diluted conditions, suggesting that activity through the oxidative TCA cycle is low. Therefore, malate production via the oxidative TCA cycle, and therefore CO₂ loss through the TCA cycle, are low.

### Balancing Cofactor and Expression Requirements to Construct Multi-Enzyme Pathways

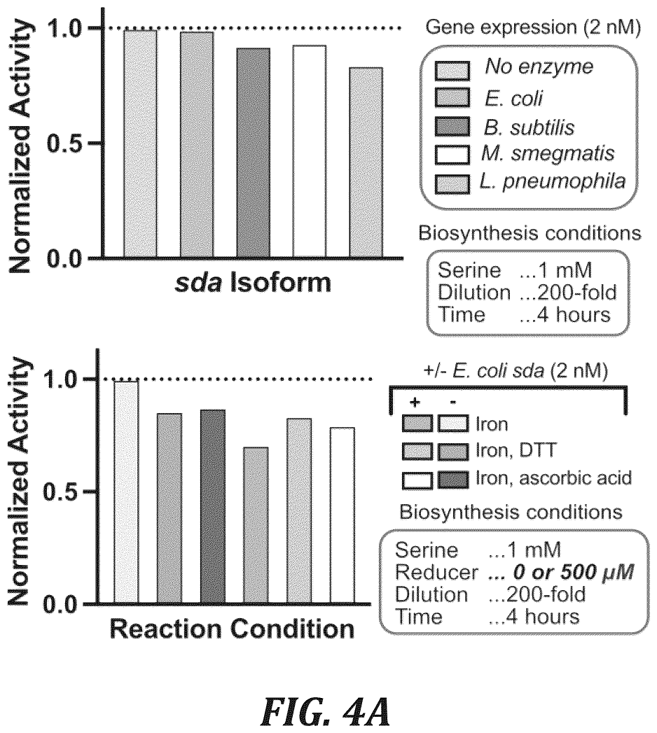

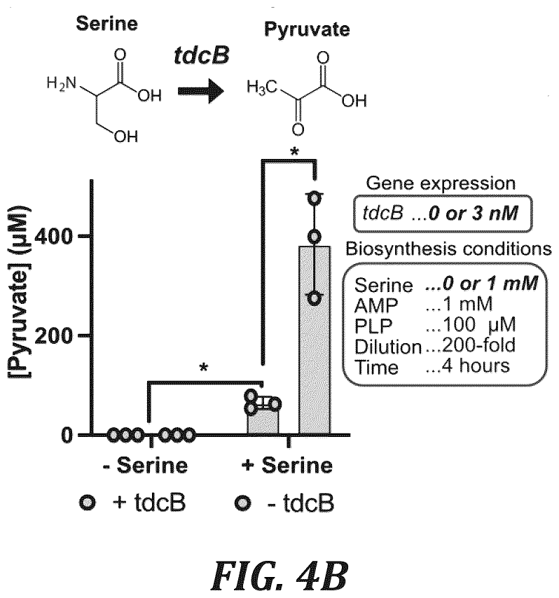

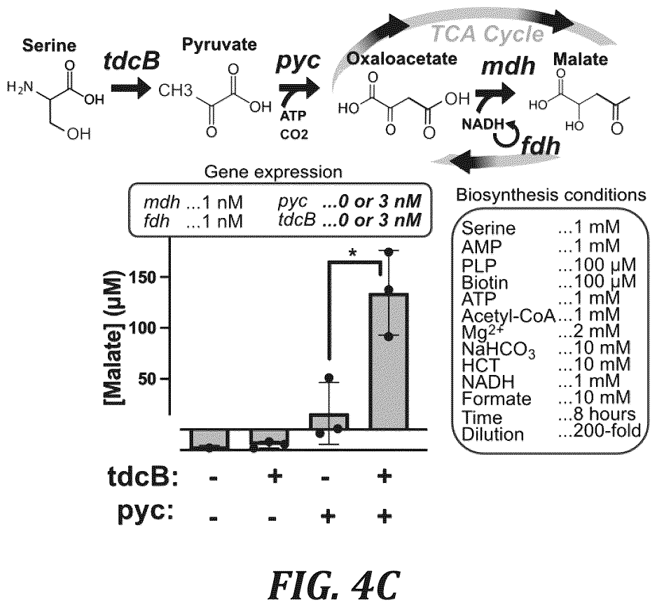

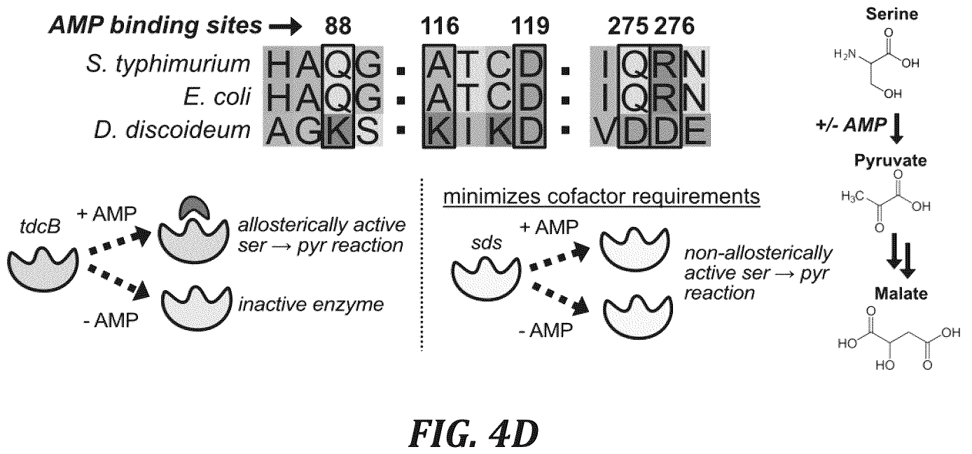

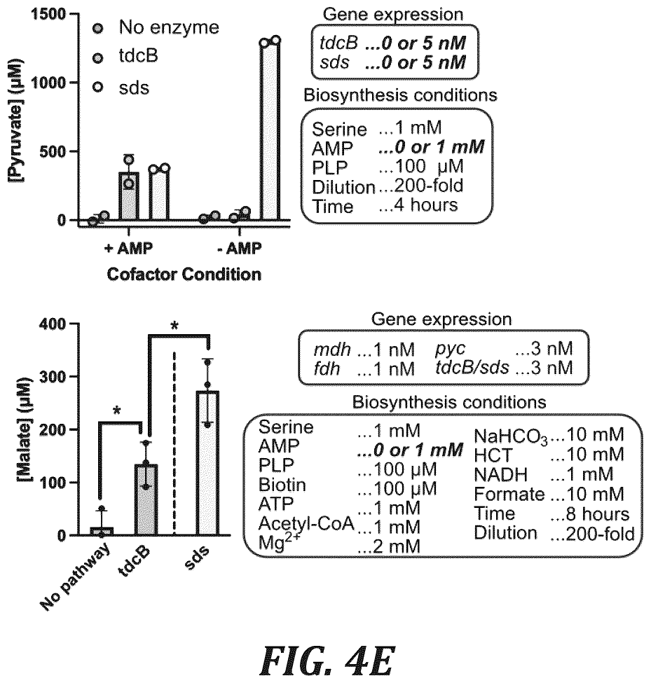

Inhibiting citrate synthase effectively blocked TCA flux, allowing malate to be produced from pyruvate through the carbon-conserving rTCA pathway. To explore approaches for producing malate from simpler substrates, the pathway was extended to start from serine, which can be synthesized from the C2 substrate glycine and the C1 substrate formate via the rGCV pathway. Serine can be efficiently converted into pyruvate through the heterologous expression of serine dehydratase (sda) in E. coli. Several isoforms of the sda enzyme from a variety of prokaryotic organisms were screened in the standard CFE conditions and observed no catalytic activity (FIG. 4A). As most prokaryotic isoforms of sda contain an oxygen-sensitive iron-sulfur cluster, enzyme function by supplementing iron and reducing agents, including dithiothreitol (DTT) and ascorbic acid at the time of expression, was attempted. However, no clear activity from any of the sda isoforms screened was observed (FIG. 4A).

The promiscuous serine to pyruvate conversion activity of a pyridoxal 5′-phosphate (PLP) dependent threonine dehydratase (tdcB) from E. coli was attempted. By adding exogenous serine into 200-fold diluted reactions, serine-dependent tdcB activity was confirmed, as tdcB expression produced 384±101 μM pyruvate from 1 mM serine, compared to 65±13 μM without tdcB expressed (FIG. 4B). In the 10-fold diluted reaction condition, no differential production of pyruvate was observed between the conditions with or without tdcB. Based on these results, the 200-fold dilution condition was employed for all subsequently described reactions.

Having demonstrated tdcB activity in a single-step transformation, integrated tdcB was integrated with the rTCA pathway to produce malate from serine. Malate production was measured in four reaction conditions with and without tdcB and/or pyc, with all conditions containing mdh and fdh. At four hours, no malate production was observed across all conditions. However, pyruvate accumulation was observed in the conditions without pyc, suggesting pyc was effectively pulling pyruvate flux forward. By eight hours, malate titers reached 135 μM when both pyc and tdcB were expressed, compared to no malate accumulation in the conditions without both enzymes expressed (FIG. 4C).

In multi-enzyme cell-free systems, the use of enzymes with numerous cofactor requirements can reduce pathway efficiency and increase costs. Incorporation of ATP-regenerating systems in CFE can reduce cofactor requirements; however, the addition of AMP as an allosteric activator for tdcB was hypothesized to interfere with ATP-dependent enzymes in the CFE. Therefore, an alternative to tdcB that was insensitive to AMP regulation was identified. Sequence motifs were extracted from tdcB known to interact with AMP. Multiple sequence alignment was used to choose other PLP-dependent enzymes that lacked these motifs and were predicted to be AMP-insensitive (FIG. 4D). From this screen, a serine dehydratase (sds) variant was selected from the soil-dwelling amoeba Dictyostelium discoideum to characterize in CFE. In the presence of AMP, the sds variant from D. discoideum produced similar levels of pyruvate as compared to tdcB from E. coli (FIG. 4E). In reactions without AMP, sds achieved 100% conversion of serine to pyruvate after only four hours, while production from tdcB was indistinguishable from the background (FIG. 4E). The ability of each isoform to convert serine to malate via the rTCA cycle was then tested. Here, it was found that sds resulted in 2-fold higher malate concentrations compared to tdcB in an eight-hour reaction (FIG. 4E). Based on these results, subsequent pathway engineering described herein was done with sds in place of tdcB.

### Connecting the Reductive TCA Cycle to Formate Assimilation Products

In a fully integrated system for carbon-conserving malate production, the combined formate assimilation, rGCV, and rTCA pathways would fix 2 CO₂ equivalents from formate and 2 from bicarbonate per malate produced. Glycine, the C2 product of the combined formate assimilation and rGCV pathways, can be converted to serine via the enzyme serine hydroxymethyltransferase (SHMT) encoded by the glyA gene from E. coli. glyA utilizes 5,10-CH₂-THF as a C1 donor to assimilate a second formate-derived carbon into glycine (FIG. 1). However, creating conditions for the rGCV pathway to run efficiently in the direction of glycine synthesis requires significant optimization of gene ratios and reaction tuning. glyA activity was prototyped and the efficiency of carbon incorporation into malate from glycine and 2 CO₂ equivalents, 1 from formate and 1 from bicarbonate, was investigated.

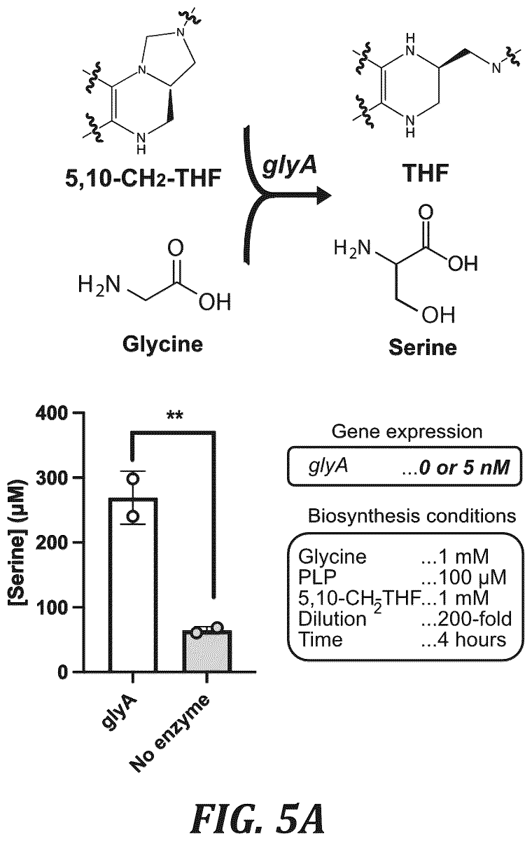

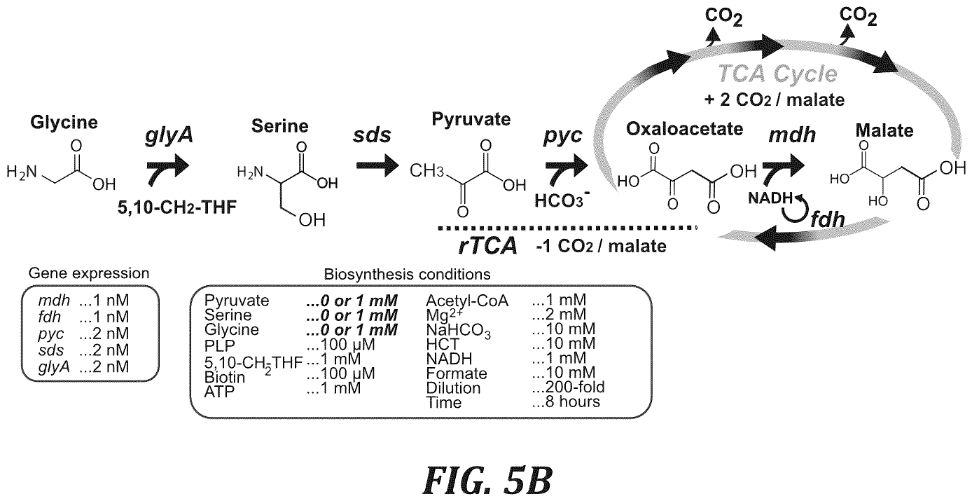

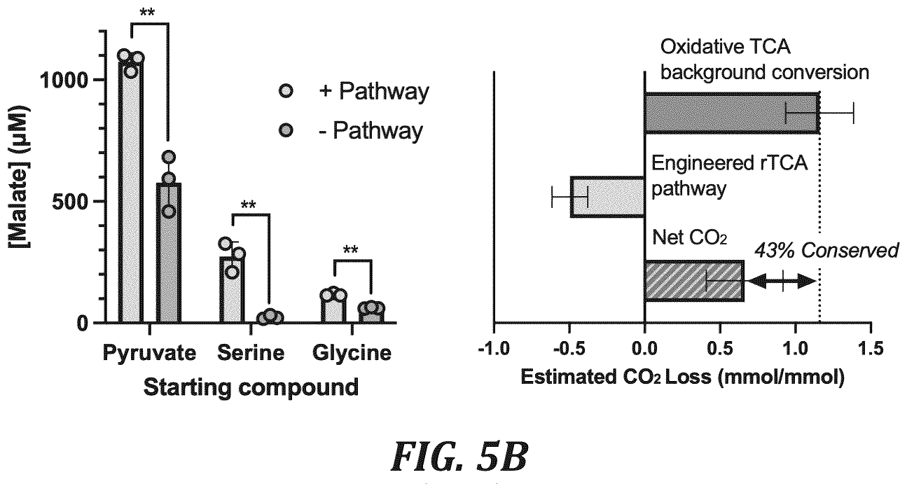

When fed with equimolar glycine and 5,10-CH₂-THF, glyA converted 27% of the glycine into serine in four hours (269±41 μM) in a 200-fold diluted cell-free reaction (FIG. 5A). By incorporating glyA and sds with the rTCA genes, pathway-dependent malate production was observed directly from C2 glycine (FIG. 5B, top). Individual starting metabolites (pyruvate, serine, and glycine), were fed to batch reactions to analyze the efficiency of malate production from each step of the pathway was observed. From pyruvate, 1075±36 μM malate in the presence of the pathway and 578±113 μM with no pathway was observed (FIG. 5B, left). When fed serine, the pathway produced 11-fold higher malate concentrations compared to the no-pathway control (273±60 μM vs. 24±8 μM) (FIG. 5B, left), suggesting the basal conversion of serine to pyruvate is low in CFE. From glycine, 117±6 μM malate was produced with-and 62±3 μM without-the pathway (FIG. 5B, left).

The amount of CO₂ conserved in the engineered system using the pathway-dependent yield was estimated from the reactions fed with pyruvate. To calculate pathway-dependent yield, the difference in malate production with and without the engineered pathway was taken. Then, to estimate conserved CO₂, a fixed reaction stoichiometry between malate produced and CO₂ equivalents conserved was assumed. Specifically, the calculations specified that for each pyruvate converted to malate, a maximum of 1 CO₂ equivalent can be fixed from bicarbonate via rTCA, and a maximum of 2 CO₂ equivalents can be released via the oxidative TCA cycle (Table 1).

Given the pathway-dependent differences in malate titers measured above, the engineered pathway conserved 497 μM of CO₂ compared to the oxidative TCA cycle when feeding pyruvate. This represents 50% of the potential conservation, assuming all of the malate accumulated above basal levels is produced through the engineered rTCA cycle (Table 1). The net result is that the introduction of the rTCA pathway increased malate yields by roughly two-fold (1075 vs. 578 μM malate) while reducing the estimated carbon loss as CO₂ from the oxidative TCA cycle by 43% (0.66 vs. 1.2 mM CO₂) (FIG. 5B, right) (Table 1). Because the basal conversion of serine and glycine to malate from the endogenous lysate metabolism is low (FIG. 5B, left), it is expected that the reduction in carbon loss remains similar when extending the engineered pathway to start from intermediates upstream from pyruvate. These calculations are meant to represent the CO₂ that is conserved relative to production through an un-engineered, plain cell-free system. The system still contains many native CO₂-releasing enzymes involved in the production of alcohols, fatty acids, and other secondary metabolites. Therefore, the analysis is focused specifically on the steps within the TCA cycle that are carbon-releasing or carbon-conserving.

TABLE 1
Carbon conserved through the rTCA pathway is calculated for
the production of malate from pyruvate. Data is shown in FIG. 5B. The calculations
assume a fixed stoichiometry for the moles of CO₂ released per mole of malate generated
through either the TCA or rTCA cycles.
Carbon conservation for malate production through the rTCA pathway

| | Through TCA | Through rTCA |
|---|---|---|
| CO₂/malate | 2 | −1 |
| (mmol/mmol) | | |
| Malate yield | .58 | 5 |
| (mmol/mmol) | | |
| Carbon loss from | 1.16 | −.5 |
| pyruvate (mmol/mmol) | | |
| Total carbon loss | 1.16 | 1.16 − . 5 = .66 |
| (mmol/mmol) | | |
| Carbon loss (% of maximum) | $\frac{1.16}{2} \times 100 = 58 \%$ | $\frac{\text{.66}}{2} \times 100 = 33 \%$ |

| | |
|---|---|
| Reduction in carbon loss | $\frac{58 - 33}{58} \times 100 = 43 \%$ |

Prototyping Cofactor Regeneration to Enable Malate Synthesis from Formate

To construct the complete pathway for the conversion of formate and glycine to malate, 7 cofactors were used (FIG. 1), 4 of which are consumed during biosynthesis (ATP, NADPH, NADH, and THF). As described above, glycine and 5,10-CH₂-THF can be used to generate malate. Here, the formate assimilation pathway for 5,10-CH₂-THF regeneration to produce malate from glycine and formate directly is prototyped. 5,10-CH₂-THF can be generated from THF and formate in three enzymatic steps catalyzed by the ATP-dependent formate-THF ligase (ftl), methenyl-THF cyclohydrolase (fch), and the NADPH-dependent methylene-THF dehydrogenase (mtdA), all from M. extorquens (FIG. 6A, left). fdh-mediated regeneration of NADH was effective for malate accumulation from the rTCA pathway. Accordingly, a polyphosphate kinase (ppk) ATP regeneration system was prototyped with a NADPH regenerating system employing fdh mutants with engineered specificity towards NADPH to regenerate cofactors consumed by the formate assimilation pathway.

To generate the biocompatible C1 donor 5,10-CH₂-THF in situ, the reductive TCA module was coupled with the three-enzyme formate assimilation pathway. In total, this represents an eight-enzyme pathway for malate production, all directly expressed from DNA in a one-pot reaction. As described herein, overexpression of the M.extorquens genes ftl, fch and mtdA for the formate assimilation pathway in the CFE improved 5,10-CH₂-THF accumulation 1.6-fold compared to lysate alone, reaching 178±35 μM from 1 mM THF and 10 mM formate after four hours (FIG. 6A).

To regenerate ATP, a polyphosphate kinase (ppk) from a previously published Erysipelotrichaceae bacterium was tested using inexpensive polyphosphate as a phosphate donor. The ppk used in this system is a Class III ppk2 enzyme, which has been shown to catalyze phosphorylation of both nucleoside mono- and di-phosphates. It was found that, when expressing ppk in CFE, 742±3 μM ATP was generated from 1000 μM AMP and 10 mM hexaphosphate in a four-hour reaction. However, when ppk was incorporated along with the biosynthetic pathway for serine to malate conversion, it was found that ATP regeneration did not improve titers over an eight-hour reaction. Therefore, it was concluded that ATP availability is not limiting flux through the rTCA pathway in these conditions. ATP regeneration in the ATP-dependent formate assimilation pathway was tested under standard conditions (1 mM ATP, 1 mM NADPH, 1 mM THF) and found that 5,10-CH₂-THF accumulation was abolished, despite the ATP regeneration system itself remaining functional. This result indicates that the ATP regeneration system introduces pathway incompatibilities in the system, and so it was excluded in future experiments.

To regenerate NADPH consumed by mtdA during formate assimilation, two NADPH-selective fdh variants for orthogonal NADH and NADPH regeneration were tested in situ. Disappointingly, no NADPH accumulation was observed in reactions fed with 1 mM NADP⁺ after screening variants from A. thaliana and Candida methylica. This may be due to poor expression or low activity of these enzymes, resulting in them not being able to compensate for native NADPH consumption in the lysate. Thus, one reaction set-up for cell-free malate production consisted of the eight-enzyme system integrating rTCA, formate assimilation, and NADH regeneration (FIGS. 1 and 6B).

The formate assimilation pathway for 5,10-CH₂-THF regeneration was applied to produce malate from C2 glycine and the C1 compounds formate and bicarbonate directly. The formate assimilation module with the rTCA cycle and fdh for NADH regeneration were co-expressed. Reactions fed with 10 mM formate, 10 mM bicarbonate, 1 mM THF, and 1 mM glycine produced 64±37 μM of malate in an eight-hour reaction (FIG. 6B), which is about half of the malate produced from 1 mM 5,10-CH₂-THF fed in directly with 1 mM glycine (117±6 μM) (FIG. 5B). This decrease in malate production is likely due to low efficiency through the formate assimilation pathway. Conversion of THF to 5,10-CH₂-THF through the formate assimilation pathway proceeded with only 18% conversion efficiency after four hours (FIG. 6A). Therefore, less 5,10-CH₂-THF was available as a substrate for glyA, which, in turn, led to decreased malate production. The reactions coupling formate assimilation, rTCA cycle and fdh also produced 67±13 μM succinate and 0.47±0.14 mM citrate as side products. With 2 CO₂ equivalents fixed per malate produced, integrating the formate assimilation pathway with the rTCA cycle process enabled incorporation of 0.13 moles of CO₂ equivalents per mole of glycine fed.

As used herein, the term “about” refers to +5% of the specified value.

### Methods

### Plasmid Preparation

Plasmids expressing pathway genes were cloned in E. coli NEB Turbo cells. All PCR amplification of genomic DNA used Phusion DNA polymerase. Primers were synthesized by IDT and gBlocks were synthesized by Twist Biosciences. Both primers and gBlocks were resuspended with nuclease-free water. Plasmid assembly was achieved using 5× In-Fusion HD mastermix (Takara). Assembled plasmids were plated onto LB-agar plates with 100 μg/mL carbenicillin. Transformed cells were grown overnight at 30° C. Single colonies were picked from plates and grown overnight in LB shaking at 30° C. with 100 μg/mL carbenicillin.

Plasmids were isolated from subcultures using a DNA miniprep kit (QIAprep Spin Miniprep Kit) and sequenced with Sanger (Genewiz inc.) or full-plasmid sequencing (Primordium) to verify correctly assembled plasmids. Plasmids were grown in culture volumes of ~100 mL to ensure adequate yields for multiple cell-free reactions. Plasmids were further purified using a PCR purification kit (Invitrogen PureLink, Cat. K310001) and eluted with nuclease-free water. Plasmid concentrations were quantified via spectrophotometry (Nanodrop 2000c, Cat. ND-2000C).

### Cell-Free Protein Synthesis Reactions

The cell-free system was acquired from Arbor Biosciences (myTXTL). The cell-free system used for an experiment was thawed on ice and pooled into a 1.5 ml Eppendorf tube, vortexed, and spun-down using a mini benchtop centrifuge to ensure homogeneity across samples. For reactions containing three or fewer genes, reactions were assembled on ice from the CFE, purified DNA, and necessary cofactors. The CFE was pipette mixed and added to each PCR tube in 7.5 L for a final volume of 10 μL. These PCR tubes were incubated overnight at 30C. For reactions involving more than three genes, plasmids and cofactors were mixed with an acoustic liquid handler robot (Echo Labcyte 525) into Labcyte 384-well destination plates (001-14555). The 384-well plates were then incubated at 30° C. overnight.

### Cell-Free Bioproduction Reactions

Cell-free bioproduction reactions were mixed in 25 μL containing 2.5 μL of CFE-expressed enzymes. CFE-expressed enzymes were diluted in 10 mM Tris pH 8 prior to adding if they were diluted beyond 10-fold in the final reaction. Bioproduction reactions were done in 50 mM HEPES pH 8. For reactions containing three or fewer enzymes, reactions were assembled by hand from the CFE-expressed enzymes and necessary substrates and cofactors. Detailed information about sample preparation can be found in Methods S3. For reactions involving more than three enzymes, enzymes and chemicals were mixed with an acoustic liquid handler robot (Echo Labcyte 525) into 96-well V-bottom plates (Costar, Cat. 3363). The plates were sealed with a foil adhesive (Thermo, AB0626) and the reactions were run for 4-8 hours at room temperature. In general, reactions containing three or fewer enzymes were run for four hours, while larger reactions were run for eight hours. This is due largely to the slow reaction progress of the pyc enzyme, which led to us allowing longer reactions when using pyc as an intermediate step. At the end of the reaction, samples were quenched with 2 volumes of acetonitrile to denature proteins. The entire sample volume was then filtered using 96-well 0.2 μm Supor membrane plates (Cytiva: 8019) and spun for 15 min at max speed. Samples were then either analyzed via LC/MS or stored at −20C until ready for analysis.

Metabolite quantification with LC/MS

Samples were analyzed via Agilent 6530 LC/Q-TOF in negative mode using a BEH Amide 50 mm column (Waters, 186004800). Standard curves were prepared by spiking known amounts of metabolites into diluted CFE and HEPES. For LC/MS, the aqueous phase was LC/MS grade water and the organic phase was 95/5 acetonitrile/water with 10 mM ammonium acetate and 0.04% v/v ammonium hydroxide. The % aqueous/organic gradient was run as follows: hold at 5/95 for 2.5 minutes, move to 33.5/66.5 over 5 minutes, 40/60 over 1 minute, hold 40/60 for 1 minute, then return to 5/95 over 1 minute. The flow rate was held at 0.5 mL/min.

### Statistics

Statistical significance was calculated using two-tailed unpaired Welch's t-tests. Asterisks in Figures indicate a statistically significant difference (*: p-value <0.05, **: p-value <0.005).

While illustrative embodiments have been illustrated and described, it will be appreciated that various changes can be made therein without departing from the spirit and scope of the invention.

1. A method for cell-free bioproduction of a chemical compound by engineering a multi-step metabolic pathway, comprising:
(a) producing one or more enzymes utilized for producing a desired chemical compound from one or more DNA templates in a lysate-based cell-free expression system;
(b) producing one or more desired chemical compounds in a cell-free metabolic engineering reaction by reacting one or more substrates with the one or more enzymes in the diluted expression system,
wherein the method further comprises at least two of the following three steps:
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) producing the one or more desired chemical compounds further comprises reactions with one or more inhibitors to substantially block activity of endogenous enzymes present in the expression system that do not produce the one or more desired chemical compounds; and
(iii) wherein producing the one or more enzymes from the one or more DNA templates comprises expression of an enzymatic cofactor regeneration system to drive the production of the one or more desired chemical compounds.

2. The method of claim 1, wherein the one or more DNA templates comprise plasmid or linear DNA.

3. The method of claim 1, wherein the lysate-based cell-free expression system is a cell lysate comprising cellular proteins and metabolites and is substantially free of cell membrane and genomic nucleic acid.

4. The method of claim 1, wherein the lysate-based cell-free expression system is an E. coli lysate.

5. The method of claim 1, wherein diluting the expression system comprises diluting from about 10-fold to about 1000-fold.

6. The method of claim 1, wherein the enzymatic cofactor regeneration system comprises expression of formate dehydrogenase (fdh) to regenerate NADH to drive metabolic flux towards the desired chemical compound by maintaining a high concentration of reducing equivalents.

7. The method of claim 1, wherein the one or more DNA templates encode formate-THF ligase (ftl), methenyl-THF cyclohydrolase (fch), methylene-THF dehydrogenase (mtdA), Mdt, GcvHPL, GcvHTP, glycine hydroxymethyltransferase (glyA), pyruvate carboxylase (pyc), malate dehydrogenase (mdh), serine dehydratase (sds), polyphosphate kinase (PPK), lipoamide dehydrogenase (Lpd), lipoate-protein ligase A (LplA), serine hydroxymethyltransferase (Shmt), serine dehydratase (Sda), PCX, formate dehydrogenase (fdh), PPT, GcvH, GcvT, GcvP, and combinations thereof.

8. The method of claim 1, wherein the one or more enzymes are formate-THF ligase (ftl), methenyl-THF cyclohydrolase (fch), methylene-THF dehydrogenase (mtdA), Mdt, GcvHPL, GcvHTP, glycine hydroxymethyltransferase (glyA), pyruvate carboxylase (pyc), malate dehydrogenase (mdh), serine dehydratase (sds), polyphosphate kinase (PPK), lipoamide dehydrogenase (Lpd), lipoate-protein ligase A (LplA), serine hydroxymethyltransferase (Shmt), serine dehydratase (Sda), PCX, formate dehydrogenase (fdh), PPT, GcvH, GcvT, GcvP, and combinations thereof.

9. A method for cell-free bioproduction of malate, comprising:
(a) producing one or more enzymes utilized for malate production from one or more DNA templates in a lysate-based cell-free expression system; and
(b) producing malate in a cell-free metabolic engineering reaction by reacting one or more substrates with the one or more enzymes in the diluted expression system.

10. The method of claim 9 further comprising one or more of steps (i) to (iii):
(i) diluting the expression system with a solvent to decrease the concentration of the one or more enzymes to improve their volumetric efficiency in a subsequent bioproduction reaction and to reduce the activity of endogenous enzymes present in the expression system relative to the one or more enzymes produced step (a);
(ii) reacting with one or more inhibitors to substantially block activity of endogenous enzymes present in the expression system that do not produce malate; and
(iii) expressing an enzymatic cofactor regeneration system to drive the production of malate.

11. The method of claim 9, wherein the one or more DNA templates encode fch, ftl, mtdA, glyA, sds, pyc, mdh, and fdh.

12. The method of claim 9, wherein the one or more enzymes are fch, ftl, mtdA, glyA, sds, pyc, mdh, and fdh.

13. The method of claim 9, wherein the one or more substrates are formate, glycine, and bicarbonate.

14. The method of claim 9, wherein the one or more inhibitors include hydroxycitrate.

15. The method of claim 9, wherein the enzymatic cofactor regeneration system provides in situ regeneration of NADH.

16. The method of claim 9, wherein malate is produced from formate, glycine, and bicarbonate in the following steps,
formate is coupled with tetrahydrofolate (THF) to provide 5,10-methylenetetrahydrofolate (5,10-CH₂-THF),
5,10-CH₂-THF transfers C1 to glycine to provide serine and recycle THF,
serine is converted to pyruvate,
pyruvate is converted to oxaloacetate with bicarbonate, and
oxaloacetate is reduced to malate using NADH.

17. The method of claim 15 further comprising regenerating NADH in the oxaloacetate reduction step with formate dehydrogenase (fdh).

18. The method of claim 15 further comprising adding an inhibitor to block the conversion of oxaloacetate to citrate.
